(19) United States
(12) Patent Application Publication
(10) Pub. No.: US 2002/0016637 A1
(43) Pub. Date: Feb. 7, 2002

(54) SOFT TISSUE FILLER

(76) Inventor: MARK A. ANTON, LAGUNA BEACH, CA (US)

Correspondence Address: MARK S ELLINGER, FISH & RICHARDSON, 60 SOUTH SIXTH STREET, SUITE 3300, MINNEAPOLIS, MN, 55402

(21) Appl. No.: 09/212,517

(22) Filed: Dec. 16, 1998

(51) Int. Cl. A61F 2/02; A61K 35/12

(52) U.S. Cl. 623/23.73; 424/572; 623/23.72

(57) ABSTRACT

The invention relates to an injectable soft tissue filler composition and methods of preparing the composition.

### TECHNICAL FIELD

The invention relates to the augmentation of facial and body surface contours with an injectable soft tissue filler composition.

### BACKGROUND OF THE INVENTION

Synthetic implants such as silicone, hydroxyapatite, and polytetrafluoroethylene, and autologous tissues such as dermis, fat, fascia, and bone have been used to augment facial and body contours of humans. Typically, material is surgically implanted through an incision. Some materials, such as small strips of polytetrafluoroethylene, cadaver fascia or cadaver skin (Alloderm®), can be placed through smaller incisions to fill small areas, including wrinkles, nasolabial folds and lips, in an office setting. Polytetrafluoroethylene and fascia implants are permanent, whereas Alloderm® is likely to be absorbed into surrounding tissues over a period of weeks and months. Autologous fat can be injected to enhance contours, but results are transient and generally not satisfying.

Cutaneous irregularities or defects, such as acne scars, wrinkles, and post-traumatic depressions are thought to result from a loss of collagen. Injection of bovine or porcine collagen, such as Zyderm®, Zyplast®, or Fibrel®, repairs the defects for about 3 to 6 months. These collagen materials are approved for intradermal injection, and may be reabsorbed by the body as they are reconstituted and re-crosslinked solutions of collagen that is not in its native form. A solution of bovine collagen and polymethacrylate beads also has been injected. The collagen in this solution is thought to serve as a vehicle to deliver the beads and is later reabsorbed, whereas the beads are encapsulated by tissue to permanently repair defects. The synthetic beads, however, possibly can erode through the skin at a later time.

### SUMMARY OF THE INVENTION

The invention is based on the discovery that an injectable soft tissue filler composition that includes a pulverized preparation of fascia in a carrier can be of great benefit to cosmetic and reconstructive plastic surgery patients. The injectable soft tissue filler composition is permanent and allows sculpting and/or enlarging various parts of the body in an easy and safe manner. The composition obviates the need for larger incisions and for a general anesthetic with its accompanying risks and costs. The compositions and methods described herein allow for soft tissue sculpting to be done in a slow, step-wise fashion to achieve the exact look that the patient and physician desire.

The invention features a method for preparing an injectable soft tissue filler. The method includes pulverizing a substantially pure preparation of fascia to form particles and suspending the particles in a carrier to provide the injectable soft tissue filler. The method further can include sterilizing the injectable soft tissue filler. The substantially pure preparation of fascia can be animal or human fascia, and can include thicker fascia layers such as tensor fascia lata. Suitable carriers include aqueous and non-aqueous carriers. The particles can be from about 0.01 mm to about 1.5 mm in diameter.

In another aspect, the invention features an article of manufacture that includes an injectable soft tissue filler composition and a container, wherein the injectable soft tissue filler composition includes a pulverized substantially pure preparation of fascia. The article of manufacture further can include a carrier. As described above, the carrier can be aqueous or non-aqueous. Suitable containers include syringes and bottles.

The invention also relates to an injectable soft tissue filler composition that includes a pulverized substantially pure preparation of fascia in a carrier. The fascia can be animal or human fascia, and can include a thick fascia layer such as tensor fascia lata. The composition can further include a pharmaceutical agent such as a local anesthetic, an analgesic, an antifungal agent, or an antibiotic.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The invention features a method for preparing an injectable soft tissue filler. The term “injectable” indicates that the soft tissue filler can be dispensed from a syringe. The soft tissue can be injected with manual pressure or with the aid of a mechanical microinjector. Such microinjectors allow the soft tissue filler to be delivered in a consistent manner. In particular, the soft tissue filler can be injected through a needle having a gauge of at least 16-18 without denaturing the fascia. The method includes pulverizing a substantially pure preparation of fascia to form particles, and solubilizing the particles in a carrier to provide the injectable soft tissue filler. As used herein, “substantially pure preparation of fascia” refers to a preparation of fascia that has been separated from components which naturally accompany it. Fascia is a fibrous membrane that covers, supports, and separates muscles. Thus, a substantially pure preparation of fascia is separated from any associated tissues such as skin or muscle. Fascia is a very dense collagen matrix containing few cells, that comes in varying thicknesses and that can be used for autografts, allografts and xenografts. For example, porcine heart valves also are of a dense collagen matrix and are routinely transferred to human hearts without reabsorption. Human cadaveric fascia can be used to strengthen joints, replace dura mater and raise eyelids. See, Crawford, J. S., Opthalmic Surgery, 8(4):31040, 1977. In thicker fascia layers, such as tensor fascia lata, striations of the fibers are seen easily and typically are cut along the fibers into smaller strips for larger implants. The relative permanence of fascia following implantation and its feel of natural tissue provides a particularly useful composition for augmenting soft tissue.

The injectable soft tissue filler described herein can enhance contours, enlarge structures and fill in depressions for cosmetic and reconstructive plastic surgery patients. For example, the injectable soft tissue filler can be used for augmenting orbital rims, malar eminences, submalar areas, chins, jawlines, mandibular angles, lips, anterior maxilla, nasal defects, sublabial creases, nasolabial lines, temporal hallows, mastoid surgery depressions, cranial burr holes and defects, dermal scar retractions, localized steroid atrophy problems, radical neck dissection depressions, nipple reconstructions, skin graft depressions, and post liposuction contour depressions. The soft tissue filler can be used to make the nasopharyngeal orifice more competent in cleft palate patients, as well as making the esophageal and other sphincters more competent. In addition, the injectable soft tissue filler can be used to enlarge penile girth, narrow vaginal introitus, and fill facial sinuses after ablation.

The soft tissue filler can be injected once, or can be injected repeatedly over a period of time to achieve the desired result. For example, a single injection of the soft tissue filler may be sufficient to repair a minor defect, whereas multiple injections over a period of weeks or months may be necessary to repair a more major defect. The soft tissue filler can be injected, for example, intradermally, subcutaneously, subdermally, and sub- and superperiosteally.

Fascia can be harvested from cadavers and animals using standard techniques. Bovine, porcine, and equine fascia are suitable for preparing the injectable soft tissue filler. Thicker fascia layers such as substantially pure tensor fascia lata are particularly useful, although thinner fascia layers are also useful. Banked fascia lata harvested from tissue donors is available from the American Red Cross, where the fascia lata is trimmed and freeze-dried over a seventy-two hour period and shipped under vacuum. Aseptic and pre-irradiated forms of the fascia lata are available. Alternatively, autologous fascia lata can be harvested using standard techniques.

Typically, fascia is found as a flexible, yet resilient, sheet of about 0.1 to about 3.0 mm in thickness, with a natural grain. The fascia can be pulverized under native conditions into particles that are, for example, from about 0.01 mm to about 1.5 mm in diameter. For example, the fascia can be processed into particles of approximately 0.03 mm to about 0.14 mm, the internal diameters of 26XV and 16XT gauge needles, respectively. Fascia can be pulverized in many different manners. In general, fascia can be subjected to freezing, heating, freeze drying/vacuum lyophilizing, tanning, stretching, pounding or compressing. For example, frozen or freeze-dried fascia can be cut into appropriate size pieces with a suitable tool, such as rotating/oscillating blades, a punching instrument, or a laser. The fascia can be fixed to a cutting surface with tension, suction, or freezing. Alternatively, the fascia can be cut into small pieces (about 5 to 10 mm) using a sharp blade. Fascia pieces can be frozen using liquid nitrogen or other solutions less than 0° C., including a dry ice/ethanol mixture. Frozen fascia pieces are brittle and can be pulverized mechanically by grinding between two surfaces, such as between a mortar and pestle. Alternatively, fascia can be pulverized by passage between two rolling drums that are separated by a defined dimension. For example, the drums can be separated by about 0.8 mm or less.

Pulverized particles of fascia can be resuspended in a sufficient amount of a carrier to produce an injectable soft tissue filler. The amount of carrier used to resuspend the particles depends on the size needle to be used, and whether a liquid, gel, or paste-like form of the composition is desired. Suitable carriers are able to prevent clumping of the fascia particles, are stable at room temperature and at 4° C., and can be aqueous or non-aqueous. Viscosity of an aqueous carrier can be increased by addition of sucrose or PEG. Non-limiting examples of aqueous carriers include saline solutions (e.g., about 0.9% sodium chloride in water), buffered saline solutions (e.g., phosphate, carbonate, bicarbonate, and cacodylate), buffered saline with about 5% to about 95% sucrose, buffered saline with about 5% to about 95% polyethylene glycol, or glycerol solutions that are at physiologic pH. For example, saline solutions containing about 20% sucrose are particularly useful for resuspending the fascia. The aqueous carrier enhances passage of the suspended fascia through needles by preventing clumping of the fascia particles.

The composition can further include a pharmaceutical agent. For example, a local anesthetic, analgesic, antifungal agent or antibiotic can be added to the composition. Pharmaceutical agents can be added to the composition by mixing with, for example, the aqueous carrier. The injectable soft tissue filler can be sterilized using known methods, including steam, ethylene oxide, and radiation.

The invention also relates to an article of manufacture including an injectable soft tissue filler composition and a container. The injectable soft tissue filler composition includes a pulverized substantially pure preparation of fascia in a carrier as described above. In particular, the carrier can be aqueous. The container can be, for example, a bottle or a syringe, and also can include a needle, or a mechanical injector in which a syringe is placed. For example, a trocar type system is particularly useful.

Alternatively, the pulverized substantially pure preparation of fascia can be lyophilized, and can be reconstituted by addition of carrier prior to use. A lyophilized preparation of fascia may be contained within a bottle in the article of manufacture. A second bottle containing a suitable carrier also may be included in the article of manufacture.

In addition, a package insert that indicates that the injectable soft tissue filler composition is useful for enhancing contours, enlarging structures, and filling in depressions for cosmetic and reconstructive plastic surgery patients also can be included.

The invention will be further described in the following example, which does not limit the scope of the invention described in the claims.

### EXAMPLE

Human fascia was rinsed in sterile water and cut into approximately twenty 1 cm square pieces. Each piece was dropped into liquid nitrogen for 20 minutes or until completely frozen. Frozen fascia pieces were transferred to a pre-chilled porcelain mortise (−80° C.). Liquid nitrogen was added to the mortise and the fascia pieces were manually ground into small pieces using a porcelain pestle. Care was taken to ensure that the liquid nitrogen did not evaporate completely. After about 30 minutes of grinding, the fascia was in the form of a crystalline powder and was stored at −80° C. until lyophilization.

Lyophilization was performed in a laboratory vacuum lyophilizer for 24 hours. The lyophilized fascia was resuspended gently with approximately 2 ml of a 20% sucrose solution in saline using a plastic pipette. The suspended fascia was loaded into a 1 cc syringe fitted with an 18 gauge needle, where it easily passed through the needle. After storage at about 4° C. for about seven days, the suspended fascia could still be passed through an 18 gauge needle

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for preparing an injectable soft tissue filler comprising pulverizing a substantially pure preparation of fascia to form particles and suspending said particles in a carrier to provide said injectable soft tissue filler.

2. The method of claim 1, wherein said substantially pure preparation of fascia comprises a thick fascia layer.

3. The method of claim 1, wherein said substantially pure preparation of fascia comprises animal fascia.

4. The method of claim 1, wherein said substantially pure preparation of fascia comprises human fascia.

5. The method of claim 2, wherein said thick fascia layer is tensor fascia lata.

6. The method of claim 1, wherein said carrier is aqueous.

7. The method of claim 1, wherein said carrier is non-aqueous.

8. The method of claim 1, further comprising sterilizing said injectable soft tissue filler.

9. The method of claim 1, wherein said particles are from about 0.01 mm to about 1.5 mm in diameter.

10. An article of manufacture comprising an injectable soft tissue filler composition and a container, wherein said injectable soft tissue filler composition comprises a pulverized substantially pure preparation of fascia.

11. The article of manufacture of claim 10, wherein said container is a syringe.

12. The article of manufacture of claim 10, wherein said container is a bottle.

13. The article of manufacture of claim 10, wherein said article of manufacture further comprises a carrier.

14. The article of manufacture of claim 13, wherein said carrier is aqueous.

15. An injectable soft tissue filler composition comprising a pulverized substantially pure preparation of fascia in a carrier.

16. The composition of claim 15, wherein said pulverized preparation of fascia comprises tensor fascia lata.

17. The composition of claim 15, wherein said composition further comprises a pharmaceutical agent.

18. The composition of claim 17, wherein said pharmaceutical agent is a local anesthetic.

19. The composition of claim 17, wherein said pharmaceutical agent is an analgesic.

20. The composition of claim 17, wherein said pharmaceutical agent is an antifungal agent.

21. The composition of claim 17, wherein said pharmaceutical agent is an antibiotic.

22. The composition of claim 15, wherein said carrier is aqueous.

23. The composition of claim 15, wherein said carrier is non-aqueous.
